(19) United States
(12) Patent Application Publication
(10) Pub. No.: US 2019/0219600 A1
(43) Pub. Date: Jul. 18, 2019

(54) Devices, Compositions, and Methods for Measuring Molecules and Forces

(71) Applicant: Emory University, Atlanta, GA (US)

(72) Inventors: Khalid S. Salaita, Dectaur, GA (US); Carol Frances Jurchenko, Lilburn, GA (US); Daniel R. Stabley, Memphis, TN (US)

(21) Appl. No.: 16/364,014

(22) Filed: Mar. 25, 2019

Related U.S. Application Data
(62) Division of application No. 14/347,095, filed on Mar. 25, 2014, now abandoned.
(60) Provisional application No. 61/540,615, filed on Sep. 29, 2011.

(51) Int. Cl. G01N 33/74; G01N 33/542; G01N 33/58; G01N 33/543; G01N 33/574

(52) U.S. Cl. CPC G01N 33/74; G01N 33/542; G01N 33/57492; G01N 33/543; G01N 33/54306; G01N 33/582

(57) ABSTRACT

This disclosure relates to compositions, devices and methods of detecting the presence of molecules and optionally quantifying forces associated with molecular interactions on the surface of cells and other lipids. In certain embodiments, devices disclosed herein can be used to detect forces through cell surface receptors. In other embodiments, the devices can be used to detect the presence or absence of molecules on cells or other particles or detect the changes in cell morphology after ligand receptor binding.

### CROSS-REFERENCE TO RELATED APPLICATIONS

This application is a division of U.S. application Ser. No. 14/347,095 filed Mar. 25, 2014, which is the National Stage of International Application No. PCT/US2012/57718 filed Sep. 28, 2012, which claims the benefit of U.S. Provisional Application No. 61/540,615 filed Sep. 29, 2011. The entirety of each of these applications is hereby incorporated by reference for all purposes.

### STATEMENT REGARDING FEDERALLY SPONSORED RESEARCH OR DEVELOPMENT

This invention was made with government support under 5R0IGM097399 awarded by the National Institutes of Health. The government has certain rights in the invention.

### BACKGROUND

The interplay between physical inputs and chemical reaction cascades coordinates a diverse set of biological processes that range from epithelial cell adhesion and migration to stem cell differentiation and immune response. The majority of these mechanical inputs are sensed and transduced through membrane receptors that mount a signaling cascade that is dependent on the mechanical properties of their specific cognate ligands. A major challenge to elucidating the molecular mechanisms of mechanotransduction lies in the development of tools that can measure forces applied to specific receptors on the cell surface. To address this challenge, two main classes of techniques have been developed. The first class employs single-molecule force spectroscopy (SMFS) methods, such as atomic force microscopy (AFM), and optical or magnetic tweezers to measure forces at specific sites on the cell surface. The inherent serial nature of SMFS methods coupled with the need for statistically significant data sets in cell biology has thus far hampered their wide-spread adoption. See e.g., Dufrene et al., Nat. Methods. 8 (2), 123 (2011); Müller et al., Nat. Chem. Biol. (2009), Vol. 5, pp. 383; Bustamante et al., Nature 2000, 1, 130-136; U.S. Pat. No. 5,992,226; and US Patent Application Publication No. 2004/0033482. Thus, there exists a need to identify improved methods.

Grashoff et al., Nature, 2010, 466 (7303), 263, discloses a tension sensor module (TSMod) in which an amino-acid elastic domain was inserted between two fluorophores that undergo efficient fluorescence resonance energy transfer (FRET). Grashoff et al. indicates that FRET is sensitive to the distance between the fluorophores and that FRET efficiency should decrease under tension. See also Iwai and Uyeda, PNAS, 2008, 105 (44), 16882; Meng et al., FEBS J., 2008, 275 (12), 3072; Rahimzadeh et al., Am. J. Physiol. Cell Physiol, 2011, 301: C646-C652. and Meng et al., Cell. Mol. Bioeng., 2011, 4 (2), 14.

### SUMMARY

This disclosure relates to compositions, devices and methods of detecting the presence of molecules and optionally quantifying forces associated with molecular interactions on the surface of cells and other lipids. In certain embodiments, the devices disclosed herein can be used to detect forces through cell surface receptors. In other embodiments, the devices can be used to detect the presence or absence of molecules on cells or other particles or detect the changes in cell morphology after ligand receptor binding.

In certain embodiments, the disclosure relates to devices comprising: a) a ligand; b) a flexible linker molecule linked to the ligand; c) a surface connected to the flexible linker; and d) a label fixed about the area of the ligand, wherein the device is configured to emit a light signal substantially based on a relative distance of the label from the surface. In certain embodiments, the label comprises a first fluorescent molecule conjugated to the ligand; and the device further comprises a second fluorescent molecule fixed about the area of the surface. In certain embodiments, one of the first and second fluorescent molecules acts to quench the fluorescence of the other fluorescent molecule.

In certain embodiments, the first and second fluorescent molecules are independently chosen from the set consisting of a dye, quantum dot, and fluorescent protein. In certain embodiments, one of the fluorescent molecules is Alexa 647 or QSY 21. In certain embodiments, the flexible linker is greater than 20 or 30 nm. In certain embodiments, the flexible linker is further connected to the surface through a polypeptide. In certain embodiments, the polypeptide is conjugated to the second fluorescent molecule. In certain embodiments, the polypeptide is streptavidin or an antibody. In certain embodiments, the ligand is a biological molecule, protein, protein fragment, nucleic acid, glycoprotein, polysaccharide, hormone, steroid, therapeutic agent, or other molecule with affinity for a protein or receptor. In certain embodiments, the surface is a particle, bead, wafer, array, well, microscope slide, transparent or opaque glass or polymer, or bottom of a zero-mode waveguide.

In certain embodiments, the disclosure relates to a system comprising devices disclosed herein wherein the device is connected to a lipid membrane comprising a receptor of the ligand. Typically, the receptor is binding the ligand causing an increase in light. In certain embodiments, the lipid membrane is a cell, liposome, micelle, or bilayer sheet.

In certain embodiments, the disclosure relates to methods comprising the steps of: a) exposing any of the devices disclosed herein to a sample suspected of containing a receptor to the ligand and b) detecting changes in the light signal. In certain embodiments, the sample suspected of containing a receptor to the ligand is a cell or bodily fluid obtained from a subject. In certain embodiments, the method further comprises the step of quantifying the light signal. In certain embodiments, the quantifying is automated on a computer. In certain embodiments, method further comprises outputting quantification results. In some embodiments, the method further comprises recording the detected changes on a computer-readable medium through a visual device such as a camera or video recorder.

In certain embodiments, the disclosure relates to methods of determining the effects of a sample compound on a cell or a lipid membrane comprising a) mixing a test compound with a system comprising any of the devices disclosed herein, wherein the device is connected to the lipid membrane comprising a receptor of the ligand; and b) detecting changes in the light signal.

In certain embodiments, devices disclosed herein may be used in microarrays and surface-based assay materials such as those used in methods of measuring molecular forces.

In certain embodiments, devices disclosed herein may be used for screening molecules of pharmacological interest for effect on cellular adhesion via specific receptors, or for effect on the process of endocytosis.

In certain embodiments, devices disclosed herein may be used in a diagnostic kit used to detect the stiffness of cancer cells, metastatic lung, breast, pancreatic cancer cells.

In certain embodiments, the disclosure relates to a device comprising a ligand connected to a linker and a label that emits a signal. The signal varies with the distance of the label from a surface. In most embodiments, the linker is attached to the surface. A system is created when the ligand attaches to a cell receptor. The cell receptor can exert a force on the device, thereby moving the position of the label with respect to the surface and changing the signal.

In some embodiments, the label can include two fluorescent molecules. These fluorescent molecules can be (independent of one another) fluorescent dyes, quantum dots, fluorescent proteins, or any other similarly fluorescent molecule. One fluorescent molecule is configured to remain fixed (i.e., does not substantially move its position) relative to the location of the surface when the cell receptor exerts a force on the device, while the other fluorescent molecule is configured to move its relative position with respect to the surface. The change in position of one fluorescent molecule with respect to the other can cause the signal to change in a quantifiable manner. In some embodiments, the two fluorescent molecules can be chosen based on properties such that the fluorescence of one molecule is absorbed by the other molecule and then the other molecule fluoresces at a different wavelength. In some embodiments, one of the fluorescent molecules can be configured to act as a quencher, absorbing the fluorescence of the other molecule, but not emitting any fluorescence. In one embodiment, one fluorescent molecule can be Alexa 647 and the other fluorescent molecule can be the quencher QSY21.

In some embodiments, the linker molecule can be flexible. Some embodiments can include a linker that is continuously flexible, exhibiting properties similar to those of a worm-like chain. In some embodiments the linker can be a polypeptide chain and in some embodiments the linker will be a polyethylene glycol polymer.

In some embodiments, the ligand can be a biological molecule. In some embodiments, the ligand can correspond with the receptor on a cell. An example of a biological ligand is epidermal growth factor (EGF). EGF corresponds to the EGF Receptor (EGFR) on a cell.

In some embodiments, the device may be attached to a backing. This backing can be any of a number of polymers, biological molecules, or laboratory equipment to which the linker is attached. In some embodiments, the backing may be a microscope slide.

The device can be configured to measure the binding force between the ligand and a receptor. To make this measurement, the device can be immersed in a solution containing receptors that correspond to the ligand. Then, the signal can be examined. In some embodiments, the signal examination can be performed using a microscope. In some embodiments, the signal is examined in an automated fashion.

In some embodiments, the signal examination is quantified. In some embodiments, the quantification is automated.

In some embodiments, the device can be included in a microarray, where a plurality of the cell detectors (either all the same embodiment or different embodiments) can be placed in a plurality of sites in order to examine multiple cell detectors at the same time.

In certain embodiment, the disclosure relates to methods of using the devices disclosed herein comprising the steps: immersing the device in a solution containing biological cells and detecting changes in fluorescence. In certain embodiments the method further comprises the step of quantifying the fluorescence. In some embodiments, the method further comprises outputting quantification results. In some embodiments, the method may further comprise recording the detected changes on a computer-readable medium through a visual device such as a camera or video recorder.

In certain embodiments, the disclosure relates to devices that comprise: a molecular linker having a first end and a second end; a ligand conjugated about the first end of the molecular linker; a first molecule conjugated about the first end; a surface conjugated to the second end of the molecular linker; and a second molecule conjugated about the surface, provided that at least one of the first or second molecules is a FRET donor and at least one of the first or second molecules is a FRET acceptor. In certain embodiments, the ligand is a biological molecule such as a protein, glycoprotein, polysaccharide, hormone, or steroid.

In certain embodiments, the molecular linker comprises ethylene glycol, hydrocarbon chain, polypeptide, or polynucleotide.

In certain embodiments, the linker is flexible, elastic or substantially non-elastic.

In certain embodiments, the first molecule is a fluorescent quencher to the second molecule.

In certain embodiments, the second molecule is a fluorescent quencher to the first molecule.

In certain embodiments, the donor and acceptor are the same, and FRET is detected by the resulting fluorescence depolarization.

In certain embodiments, FRET can be detected by the appearance of sensitized fluorescence of the acceptor or by quenching of donor fluorescence. Nonfluorescent acceptors such as dabcyl and QSY dyes are contemplated.

In certain embodiments, the first or second fluorescent molecule is a dye, quantum dot, or protein. In certain embodiments, the donor or acceptor molecule is selected from xanthene derivatives: fluorescein, rhodamine, Oregon green, eosin, Texas red, and Cal Fluor dyes, cyanine derivatives: cyanine, indocarbocyanine, oxacarbocyanine, thiacarbocyanine, merocyanine, and quasar dyes, naphthalene derivatives (dansyl and prodan derivatives), coumarin derivatives, oxadiazole derivatives: pyridyloxazole, nitrobenzoxadiazole and benzoxadiazole, pyrene derivatives: cascade blue, oxazine derivatives: nile red, nile blue, cresyl violet, oxazine 170, acridine derivatives: proflavin, acridine orange, acridine yellow, arylmethine derivatives: auramine, crystal violet, malachite green, tetrapyrrole derivatives: porphin, phthalocyanine, bilirubin, a CF dye (Biotium), a BODIPY (Invitrogen), a Alexa Fluor such a fluorophore is Alexa Fluor 350, Alexa Fluor 488, Alexa Fluor 546, Alexa Fluor 555, Alexa Fluor 568, Alexa Fluor 594, Alexa 647 (Invitrogen), a DyLight Fluor (Thermo Scientific, Pierce), an Atto and Tracy (Sigma Aldrich), a FluoProbes (Interchim), a MegaStokes Dye (Dyomics). QSY21.

In certain embodiments, the FRET donor acceptor pairs may be a fluorescein and a tetramethylrhodamine, IAEDANS and a fluorescein; EDANS and a dabcyl, a fluorescein and a fluorescein, a BODIPY FL and a BODIPY FL, a fluorescein and a QSY 7, QSY 9 dyes, QSY 21, or QSY 35, an Alexa Fluor and a QSY 7, QSY 9, QSY 21, or QSY 35 dyes.

In certain embodiments, the disclosure relates to a system comprising a device disclosed herein and a lipid structure comprising a receptor to the ligand. In certain embodiments, the lipid structure is a cell, liposome, micelle, or bilayer sheet. In certain embodiments, the receptor is membrane bound receptor.

In certain embodiments, the disclosure relates to a method that includes: providing a device immersed in a solution containing biological cells, the device including a) a ligand; b) a flexible linker molecule linked to the ligand; c) a surface connected to the flexible linker; and d) a label fixed about the area of the ligand, wherein the device is configured to emit a light signal substantially based on a relative distance of the label from the surface; and detecting changes in the light signal. In some embodiments, method includes a step of quantifying the fluorescence. In certain embodiments, the method includes a step of generating a force quantification map.

In certain embodiments, the methods disclosed herein is performed by a computer having a memory and a processor.

### BRIEF DESCRIPTION OF THE DRAWINGS

FIGS. 1A-D illustrate the design and response of an EGFR tension senor.

FIG. 1A shows a schematic of the EGF-PEGₓ (x=12, 24, or 75) tension sensor, comprised of a PEG polymer of length x that is flanked by fluorescently labeled (Alexa Fluor 647) EGF ligand and a biotin moiety for surface immobilization via streptavidin capture. EGF crystal structure adapted from Protein Data Bank (identifier IJL9). Residues in red in the crystal structure represent lysine and the N terminus, which are the available sites for PEG and fluorophore modification.

FIG. 1B shows a schematic of the mechanism of sensor function—it is not intended that certain embodiments of the disclosure be limited by any particular mechanism. When EGFR exerts a force on its ligand, the flexible PEG linker extends. The displacement of the EGF ligand results in an increase in the measured fluorescence intensity, thus reporting the transmission of mechanical tension through the EGF-EGFR complex. hv, emission of a photon.

FIG. 1C shows representative brightfield, reflection interference contrast microscopy (RICM) and EGFR tension sensor TIRF response of HCC1143 cells plated onto sensor surfaces at 37° C. for the indicated time points (t represents the start of imaging). Images on the bottom show magnification of the boxed regions. Colored line scans represent 34 pixel profiles through the indicated region; the color of each line corresponds to the graph shown below each set of frames. The arrows highlight fluorescent spots that persisted for 90 s, 60 s and 30 s. Top scale bar, 20 μm; bottom scale bar, 4 μm. Fluorescence intensity is given in arbitrary units (a.u.).

FIG. 1D shows histograms of the areas (n=82) and the durations (n=68) of fluorescent points under a cell that was observed for 10 min.

FIGS. 2A-2E show data and pictures for characterization and quantification of the EGFR tension senor.

FIG. 2A shows the role of the flexible linker (alkyl, 2.2 nm or PEG₇₅, 26 nm) and the quencher in the EGFR tension sensor response. Error bars, s.e.m. (n=77 cells).

FIG. 2B shows representative brightfield, reflection interference contrast microscopy (RICM), and EGFR tension sensor response (epifluorescence (epi) 640 nm) channels for cells treated with latrunculin B (LatB) or control (DMSO). Scale bar, 5 μm.

FIG. 2C shows measured EGF force response (normalized fluorescence intensity) between LatB-treated (n=33 cells) and untreated (n=32 cells). Error bars, s.e.m.

FIG. 2D shows representative dual channel TIRF microscopy images of a CLC-eGFP-transfected cell engaged to the force-sensing surface. Overlay channel shows colocalization of CLC-eGFP and the EGF-force response. Scale bar, 5 μm.

FIG. 2E shows representative brightfield, RICM, and fluorescence response for a cell engaged to an EGF-PEG₂₄ force sensor surface. The sensor fluorescence response was converted into a force map by using the extended WLC model for PEG₂₄. Scale bars, 10 μm (3.2 μm in the magnified image).

FIG. 3A illustrates the fabrication of a glass surface-functionalized force sensors embodiment. Schematic describing the steps used to generate the force biosensors.

FIG. 3B illustrates molecular structures of the reactive NHS esters of QSY 21 and Alexa 647.

FIG. 4A shows a method for generating force maps. Note that the false-color intensity values represent an ensemble average force for each pixel, and that this is the lower bound of the applied force. Scale bar is 3.2 μm.

FIG. 4B shows an example of a system configured to generate force maps.

FIG. 5A illustrates schematic of the integrin force sensor, which is comprised of a PEG polymer flanked by a peptide and quencher at one terminus, and a fluorescent streptavidin protein at the other terminus. Mechanical tension applied through integrins extend the PEG linker and increase fluorescence.

FIG. 5B shows the preparation of the cRGDfK(C) peptide, QSY-21 quencher functionalized integrin mechanophore conjugate.

FIG. 6A shows a plot of the measured streptavidin surface density and the RGD-peptide quenching efficiency as a function of the ratio of APTES to mPEG silane.

FIG. 6B shows representative fluorescence images of each surface, and the mean streptavidin intermolecular distance. Scale bar is 10 μm.

FIG. 7A shows brightfield, RICM, and integrin tension response for cell that is spreading over a cRGD mechanophore surface (density=4600 streptavidin/μm²). (FIG. 7B shows force quantification on time-lapse series of images from the noted region of interest (red box) (t represents the start of imaging).

FIG. 7C shows the sensor fluorescence response was converted to a force map by using the extended worm-like chain model for PEG₂₃. (

FIG. 7D shows line scans represent profiles through the indicated region as a function of time.

FIG. 7E shows plot of the translocation of the elongated force region as a function of time.

FIG. 8A shows representative cell response on cRGD mechanophore surface (density=520 streptavidin/μm²).

FIG. 8B shows force quantification analysis of images at different magnification levels.

FIG. 8C shows force quantification analysis of images at different magnification levels.

FIG. 8D shows force quantification analysis of images at different magnification levels.

FIG. 8E shows a line scan analysis as a function of time.

FIG. 8F shows a line scan analysis as a function of time.

FIG. 9A shows representative images of co-localization between vinculin, f-actin, and the integrin force response in cells that were grown on the cRGD-mechanophore surface (4600 streptavidin/μm²) for 1 hr, and then fixed and immuno-stained.

FIG. 9B shows line plot analysis of identical regions within each channel shows colocalization in some areas. Scale bar is 10 microns.

FIG. 10 illustrates an embodiment wherein the fluorophore, PEG linker, quencher, streptavidin, biotin, antibody, and surface are connected from top to bottom respectively, in a method for determining flow shear force. The top illustrates no flow with no shear force, and the bottom illustrates the effects of movement due to the shear force on the device.

### DETAILED DESCRIPTION

This disclosure relates to compositions, devices and methods of detecting the presence of molecules and optionally quantifying forces associated with molecular interactions on the surface of cells and other lipids. In certain embodiments, devices disclosed herein can be used to detect forces through cell surface receptors. In other embodiments, the devices can be used to detect the presence or absence of molecules on cells or other particles or detect the changes in cell morphology after ligand receptor binding.

In certain embodiments, a fluorescence-based system may be used for detecting, visualizing and potentially measuring external cellular forces or cell/cell interactions in live cells. In certain embodiments, the disclosure relates to a device comprising a platform-bound ligand fused to two molecular entities: a fluorophore and a quencher are separated by a polyethylene glycol linker. In the absence of any binding, the fluorophore ligand conjugate is in close proximity to the quenching signal, and there is no fluorescence. Upon binding to a receptor or other interacting protein, the fluorophore ligand conjugate is pulled away from the platform by these proteins, thereby separating them spatially from the quencher, activating fluorescence. The further the two are separated, the brighter the signal becomes. The strength of signal can also be correlated to the force exerted, allowing one to obtain a measure of the force exerted by the receptor on its ligand, a measure of the force of an interaction. To obtain this measurement, one can utilize software that takes the images or video and converts them into a force map, allowing users to detect the forces of this interaction anywhere in the cell.

In certain embodiments, the fluorophore ligand conjugate is replaced with a quencher ligand conjugate. The fluorophore is concurrently connected near the surface of the platform. Upon binding to a receptor or other interacting protein, the quencher ligand conjugate is pulled away from the platform by these proteins, thereby separating them spatially from the quencher, activating fluorescence near the surface of the platform.

In certain embodiments, the system may be used to detect cancer cells. Malignant cancer cells are typically “softer” than normal cells, as measured by their resistance to an externally applied force. Also of note is that different types of cancer have differing resistances; thus, in one embodiment, the disclosure contemplates the use of systems disclosed herein to create a cancer diagnostic based upon the resistance signature of a cell or tissue.

### Terms

As used herein, the term “ligand” refers to an organic molecule, i.e., substantially comprised of carbon, hydrogen, and oxygen, that binds a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are binding partners. For example, a glycan may be expressed on a cell surface glycoprotein and a lectin may bind the glycan. As the glycan is typically smaller and surrounded by the lectin during binding, it may be considered a ligand even though it is a receptor of the lectin binding signal on the cell surface. In another example, a double stranded oligonucleotide sequence contains two complimentary nucleic acid sequences. Either of the single stranded sequences may be consider the ligand or receptor of the other.

As used herein, a “flexible linker” refers to a molecular arrangement that connects molecular entities through covalent bonds and has the ability to collapse and expand in an aqueous solution within the range of forces exerted by ligands and receptors. In certain embodiments, the flexible linker is a polyethylene glycol polymer but other polymers are also contemplated such as, but not limited to, co-polymers, polypropylene glycol, hydrocarbon chains containing ethers, esters, and amides, and the like. Additional contemplated examples are polyglycine or other polypeptides with a majority of glycine amino acids, such as those containing a repeating proline, polyglycolic acid, or alkyl chains comprising with one, two, three, or more ester groups.

As used herein, the term “surface” refers to the outside part of an object. The area is typically of greater than about one hundred square nanometers, one square micrometer, or more than one square millimeter. Examples of contemplated surfaces are on a particle, bead, wafer, array, well, microscope slide, transparent or opaque glass, polymer, or metal, or the bottom of a zero-mode waveguide. A “zero-mode waveguide (ZMW)” refers to a confined structure or chamber located in an opening, e.g., hole, of a metal film deposited on a transparent substrate. See Levene et al., Science, 2003, 299:682-686. The chamber acts as a wave guide for light coming out of the bottom of the opening. The openings are typically about 150-50 nm in width and depth. Due to the behavior of light when it travels through a small aperture, the optical field decays exponentially inside the chamber. Thus, fluorescent molecules will lose fluorescence as they move away from the bottom of the chamber.

As used herein, a “label” refers to any molecular arrangement that can produce a light signal or can be modified to produce a light signal. A typical embodiment is a label that fluoresces, i.e., a fluorescent label; however, other labels are contemplated. For example, the label may be an epitope that can be recognized by a fluorescent binding moiety, e.g., antibody conjugated to a fluorescent dye.

A “subject” refers to a mammal such as a human being, livestock, or domestic pet.

### Fluorescent Sensors and Preparation

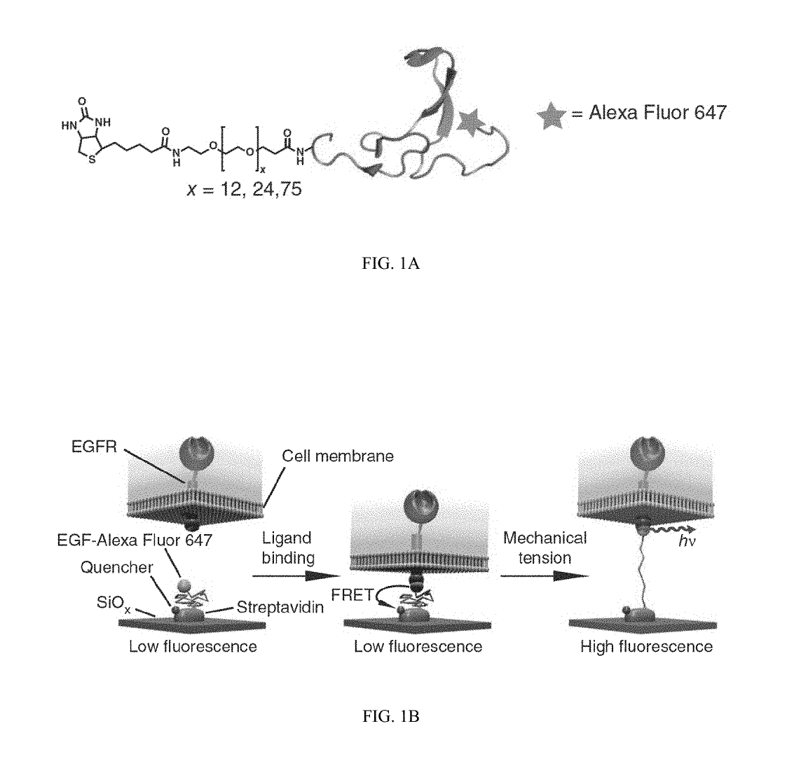

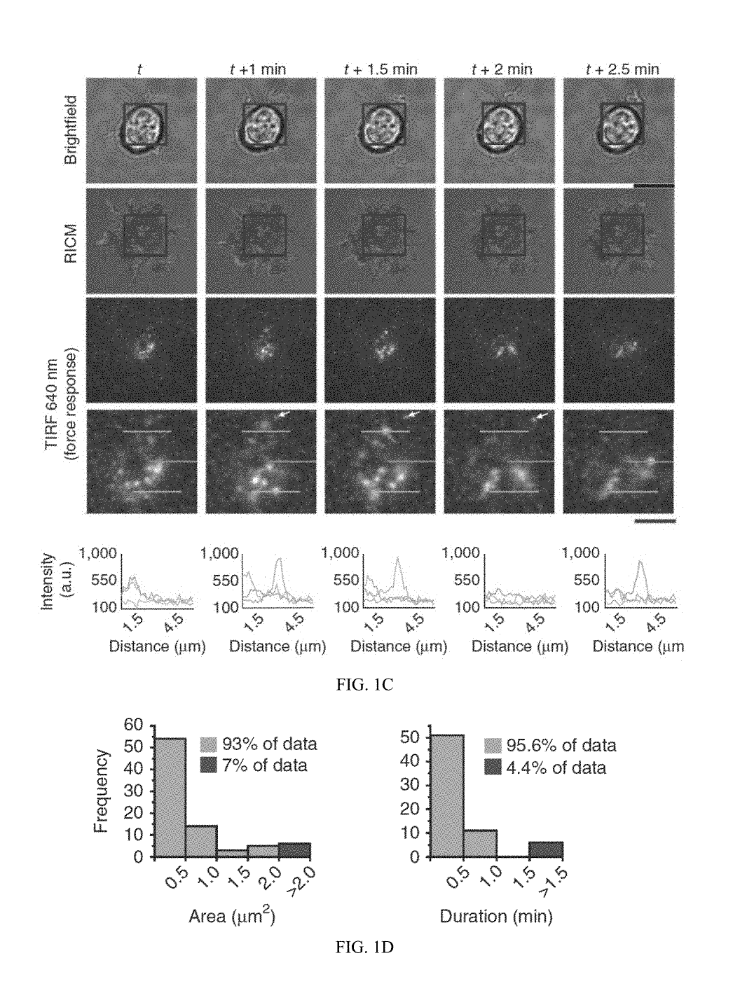

In one embodiment, the tension sensor is prepared by the processes illustrated in the Figures. In certain embodiments, the disclosure contemplates a device comprising a molecular-tension sensor that can be used, as well as be configured to, to spatially and temporally map forces exerted by cell-surface receptors. In certain embodiments, the sensor includes a flexible linker that is covalently conjugated to a biological ligand at one terminus and anchored onto a surface (via a biotin-streptavidin interaction) such that mechanical forces do not result in sensor translocation (FIGS. 1A and 1B). In certain embodiments, the sensor includes a flexible linker that is covalently conjugated to a biological receptor for a cell surface maker at one terminus and anchored onto a surface (via a biotin-streptavidin interaction) such that mechanical forces do not result in sensor translocation.

In one embodiment, a linker comprised of a polyethylene glycol (PEG) polymer is contemplated because of its unique properties that include: (i) reversible force-extension curves (ii) biocompatibility and (iii) minimal nonspecific interactions with other biomolecules. In certain embodiments, one can functionalized the ligand and the surface with fluorophore and quencher molecules, respectively; or one can functionalized the ligand and the surface with quencher and fluorophore molecule, respectively. Although certain embodiments are not intended to be limited by any particular mechanism, cellular forces exerted on the ligand extend the linker from its relaxed conformational state and remove the fluorophore from proximity to the quencher, thus resulting in increased fluorescence intensity and providing a signal to map mechanical tension transduced through specific receptor targets (FIG. 1B).

### Visualizing Mechanical Tension Across Membrane Receptors

The approach can be used to map forces with single-molecule spatial resolution and high temporal resolution in living cells. In this embodiment, this method utilizes a conventional fluorescence microscope.

In one experiment, a tension sensor was used to map forces associated with initial uptake and trafficking of the epidermal growth factor receptor (EGFR) upon binding to its cognate ligand. The EGFR pathway has important roles in cell survival, proliferation and differentiation, and internalization is an important regulatory component in the normal physiology of this pathway; it is one of the most widely studied experimental systems for investigating ligand-induced receptor endocytosis. Still, fundamental questions about the role and even the existence of forces in shuttling the receptor from the cell membrane to endosomal compartments remain. It seems rational to conclude that the process of endocytosis requires the application of a force to transport the EGFR-EGF complex, but specific evidence is thus far lacking.

Tension sensors were synthesized that present the EGF ligand and can be used to specifically measure force transmission through the EGFR. To characterize the conformation of the sensor in the resting state (in the absence of cellular forces), the EGF-PEG conjugate was tethered to a fluid supported lipid bilayer. The supported lipid bilayer surface provides a well-controlled biomimetic environment in which the protein density can be quantitatively measured and tuned. The sensors are homogeneously displayed on the laterally mobile supported lipid bilayer surface as indicated by fluorescence recovery after photobleaching. Quantitative fluorescence resonance energy transfer (FRET) efficiency measurements showed that the sensor conjugates adopted a condensed mushroom-like conformation with the EGF located 5.5±0.1 nm, 5.2±0.2 nm and 7.0±0.2 nm (mean±s.e.m., n=3) from the surface for the EGF-PEGx conjugates, where x=12, 24, and 75 monomer units, respectively. These distance values indicate that the EGF-PEG24 and EGF-PEG75 linkers adopt their predicted Flory radii. Consequently, the resting-state structures of the EGF-PEG75 and EGF-PEG24 sensor conjugates were at about 25% and about 57% of their full contour lengths, respectively, which implies that the fluorescence intensity is expected to increase considerably as the PEG linkers are fully extended. Although the conformation of PEG polymers in solution is temperature- and solvent-dependent, the equilibrium conformation of the force sensor was not appreciably altered at physiological conditions (37° C. and 1× phosphate-buffered saline (pH 7.4)). Therefore, these data, along with experimental and theoretical literature precedent investigating the force extension of PEG polymers and their protein conjugates, predict that the dynamic range of the EGF-PEG force sensors directly depends on the length of the PEG linker. For example, the dynamic range of EGF-PEG24 conjugates is about 0-20 pN, and greater than 95% of the maximum fluorescence intensity will be observed with the application of a 20 pN force. This range is compatible with the range of forces inherent to many biological processes.

### Detecting Cancer Cells

Atomic force microscopy (AFM) may be used to measure the stiffness of various cancer cells because metastatic lung, breast, and pancreatic cancer cells are on average significantly softer than benign cells. AFM is not an inherently high-throughput system. Fluorescence sensors disclosed herein may be used to make the same or similar determinations of cellular stiffness with detection of piconewton forces, and can measure many cells in a much shorter period of time than AFM techniques making it a potentially more facile diagnostic technique

When immortalized human breast cancer cells (HCC1143) were mixed with the EGFR tension sensor surface, receptors expressed in the cell membrane bound to their cognate ligands. Within 20-30 min of cell spreading, transient and localized increases in fluorescence intensity were observed via time-lapse total internal reflection fluorescence (TIRF) microscopy, which exclusively probes molecules within 150 nm of the substrate (FIG. 1C). The bright spots were diffraction-limited (FIGS. 1C and 1D), indicating that the observed events were localized to punctate points that experience mechanical tension. Additional analysis revealed that the localized increases in fluorescence were short-lived, seldom persisting longer than 30 s, and that there was a range of lifetime distributions for points across the cell-substrate contact plane (FIGS. 1C and 1D). The fluorescence intensity at these spots then returned to the background amount, indicating that the fluorophore-labeled EGF remains bound to the sensor surface. Photobleaching was not observed under these time-lapse imaging conditions during the first 20-30 frames. The recovery of the fluorescence intensity to the background level after the transient increase may be a consequence of ligand-receptor dissociation or diminished cellular pulling. The mechanism of complete internalization is most likely stalled because the ligand is tethered to the substrate, and thus the measured mechanical forces are associated with the initial steps of ligand uptake.

A wide array of adhesion receptors may interact with the underlying substrate. The specificity of the tension sensor to EGFR was tested using three sets of control experiments. First, bovine serum albumin (BSA) force-sensor conjugates were synthesized and plated cells on these substrates. The BSA conjugates under the cells displayed no fluorescence response as detected by TIRF imaging 30 min after plating. Second, cells with were pretreated 1.7 nM soluble EGF for 5 min, then the cells were plated on the EGF force-sensor surfaces. An optical response was not observed. Finally, to determine the role of an apposed ligand in the specificity of the force response, a cyclic Arg-Gly-Asp (RGD) peptide ligand was incorporated into the BSA-force sensor surface. Unlike the first two controls, cells strongly engaged these surfaces, as indicated by reflection interference contrast microscopy imaging, but the observed fluorescence response was negligible. Taken together, these experiments confirmed that the measured responses were specific to force transmission through the EGFR.

### Linker Size

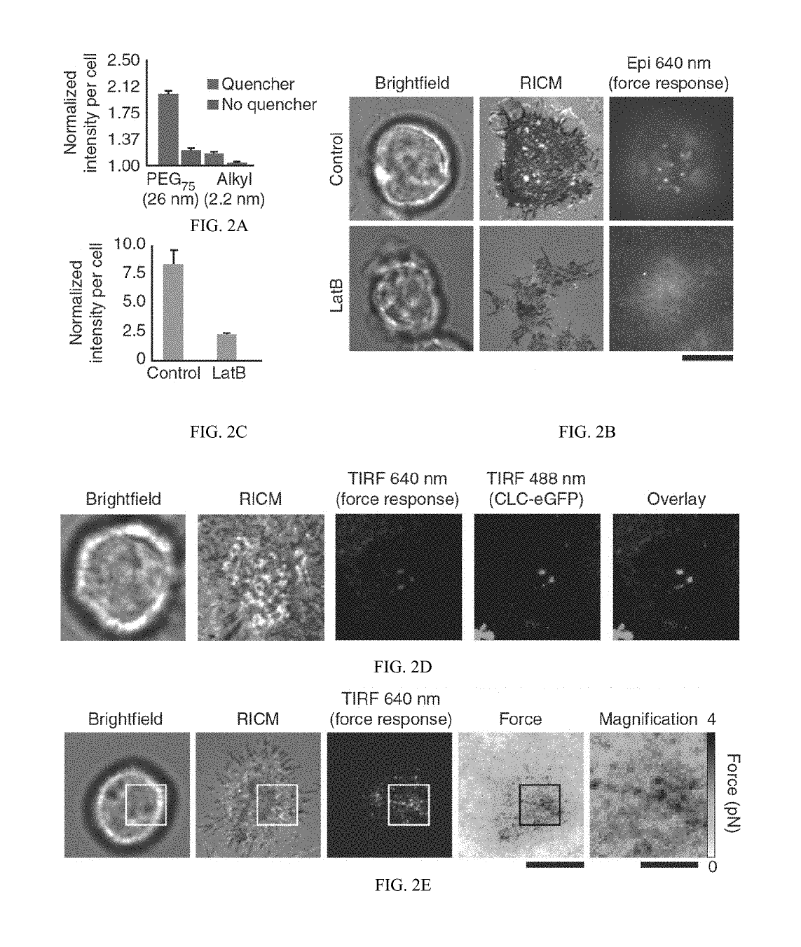

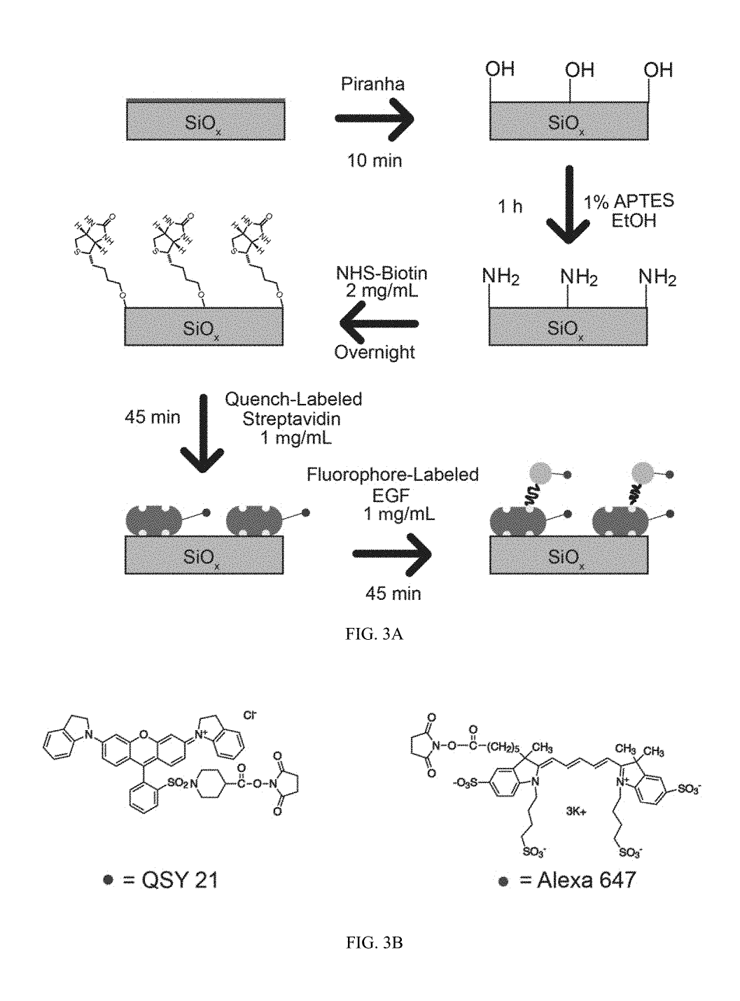

To examine the role of the PEG linker and the specific fluorophore (Alexa Fluor 647) and quencher (QSY 21) pair (Förster radius, R⁰=6.9 nm according to the manufacturer) in the observed fluorescence response, cell-tension measurements were performed with sensors displaying short linkers (contour length of 2.2 nm) or with sensors that lacked the quencher tags. In these experiments, the force response was quantified in single cells and normalized it to the background signal. Experiments with sensors containing a 2.2-nm linker showed minimal response when compared to the 26-nm PEG75 linkers (FIG. 2A). Similarly, sensors that lacked the quencher did not exhibit a notable fluorescence increase (FIG. 2A). To eliminate the possibility that direct ligand-receptor binding may lead to sensor response, EGF force-probe surfaces were treated with a monoclonal EGF antibody. This treatment did not result in a sensor response. To ensure that the biological activity of the EGF ligand was not influenced by the length (flexibility) of the different linkers, cells were immunostained with an antibody to phospho(p)Tyr1068 of EGFR to measure the relative activation. Single-cell fluorescence analysis did not indicate a marked difference in immunostaining between cells activated with tension-sensor surfaces that used 2.2-nm or 26-nm linker contour lengths, thus showing that cells were similarly activated. These experiments indicate that the tension sensor works with a flexible linker that is appropriately matched to the Förster radius of the dye pair.

### Clathrin-Mediated EGF Internalization

EGFR endocytosis is thought to primarily proceed through an internalization pathway that is mediated through the cytoskeleton and clathrin-coated pits. To look for evidence for the role of the cytoskeleton in mechanotransduction, cells were treated with latrunculin B, a cytoskeletal inhibitor that targets the assembly of F-actin. This led to a 70% reduction in sensor response, indicating that physical tension is dependent on proper function of the cytoskeleton (FIGS. 2B and 2C). To confirm that mechanical force is associated with clathrin-coated pit invagination, the HCC1143 cells were transiently transfected with a construct encoding clathrin light chain—enhanced GFP (eGFP) (CLC-eGFP). Using live-cell dual-channel TIRF microscopy the association of CLC-eGFP with the EGFR tension sensor was measured. Diffraction-limited bright spots in both fluorescence channels were observed (FIG. 2D). Taken together, the average lifetimes and dimensions of the punctate points along with actin-dependence and clathrin-colocalization data all indicate that the mechanical pulling events are consistent with a clathrin-mediated EGF internalization mechanism.

### Quantification

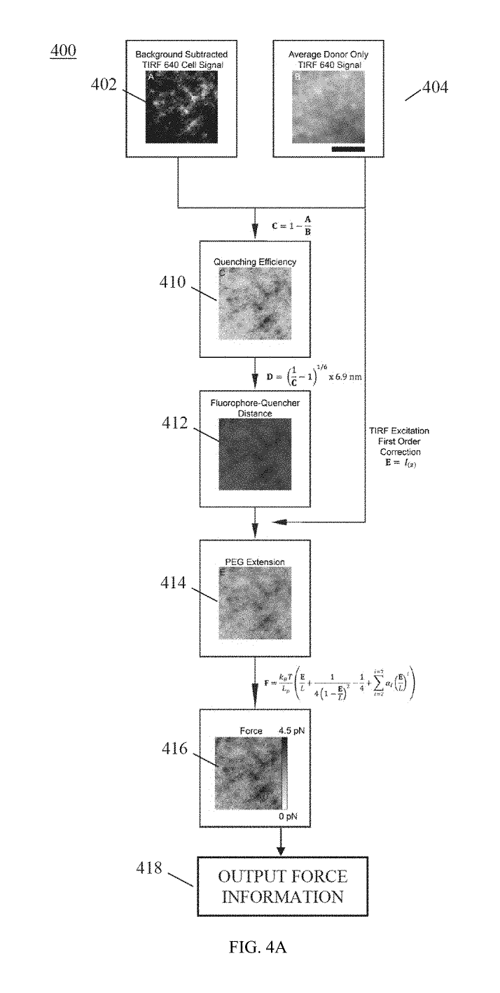

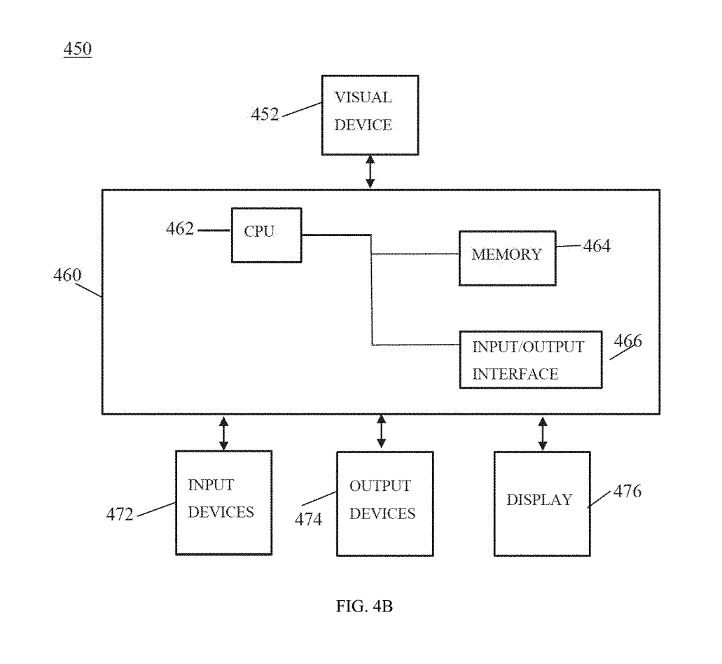

The tension sensor design allows for quantification of the magnitude of the applied force needed to extend the PEG linker from its resting state. The physical extension of the linker can be determined from the FRET relation and the displacement can be used to estimate the mechanical tension using the extended worm-like chain (WLC) model. See FIG. 4A; Oesterhelt et al., New J. Phys., 1, 6.1-6.11 (1999); Kienberger et al., Single Molecules, 1, 123-128 (2000); and Bouchiat et al. Biophys. J. 76, 409-413 (1999), all hereby incorporated by reference in their entirety.

This conversion is possible because PEG is a well-behaved polymer whose force-extension profile experimentally fits the extended WLC with high accuracy in 1×PBS buffer. Monolabeled EGF-PEG24 conjugates were used because of their broad dynamic range for force quantification. A representative force map was generated for a cell that engaged the EGF tension sensor for 30 min (FIG. 2E). The punctate fluorescent regions showed a peak force value of approximately 4 pN, which represents the lower-bound ensemble average force applied by the EGF receptor on that area.

FIG. 4A shows an example of a method 400 for quantifying forces detected by the sensor. It will be understood that forces may be quantified by any other methods.

The method 400 may include a step of receiving images. In some embodiments, the method 400 may include a step 402 of receiving a background subtracted TIRF 640 image (A) and a step 404 of receiving a composite donor only image (B). The composite donor only image may be an average over any number of regions of the donor only sample. In some embodiments, for example, the example shown in FIG. 4A, the composite donor only image can be an average of the signal over five regions of the donor only sample.

The method 400 may further include a step 410 of generating a quenching efficiency map (C). The quenching efficiency map (C) may be generated by using, for example, equation (3) in the examples and methods section below. The quenching efficiency map (C) may be generated by dividing the background subtracted TIRF 640 image (A) by a composite donor only signal image (B) to generate a quenching efficiency image map (C).

Next, a distance map (D) may be generated (step 412). The quenching efficiency map (C) may then be converted to a distance map (D) using the FRET relationship, for example, by applying equation (4) in the examples and methods section below.

In some embodiments, this distance map (D) can be then used to perform a first order correction for TIRF excitation intensity falloff (see equations 5 and 6 in the examples and methods section).

Next, in some embodiments, the z-extension of PEG may be mapped (E) in step 414, for example, after the dimensions of EGF, streptavidin, and the resting state of the polymer were subtracted out (e.g., see the examples and methods section).

Next, the force(s) may be determined (step 416). The extension image (E) can be converted to force (F), for example, using an extended WLC model (see equation 7 in the examples and methods section). Any quantitative format may be generated to present the force(s). In some embodiments, a quantitative force map may be generated in step 416.

In some embodiments, the determined force(s) may be outputted (step 418). In some embodiments, the outputting may include displaying, printing, storing, and/or transmitting the determined force(s), for example, as a quantitative force map. In some embodiments, the determined force(s) may be transmitted to another system, server and/or storage device for the printing, displaying and/or storing.

The methods of the disclosure are not limited to the steps described herein. The steps may be individually modified or omitted, as well as additional steps may be added.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

FIG. 4B shows an example of a system 450 that may be used to quantify forces detected by the sensor according to embodiments. The system 450 may include any number of modules that communicate with other through electrical or data connections (not shown). In some embodiments, the modules may be connected via a wired network, wireless network, or combination thereof. In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radio frequency network, or another similarly functioning wireless network.

Although the modules of the system are shown as being directly connected, the modules may be indirectly connected to one or more of the other modules of the system. In some embodiments, a module may be only directly connected to one or more of the other modules of the system.

It is also to be understood that the system may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the system may be time synchronized. In further embodiments, the system may be time synchronized with other systems, such as those systems that may be on the medical and/or research facility network.

The system 450 may optionally include a visual device 452. The visual device 452 may be any visual device configured to capture changes in light and/or fluorescence. For example, the visual device may include but is not limited to a camera and/or a video recorder. In some embodiments, the visual device may be a part of a microscope system. In certain embodiments, the system 450 may communicate with other visual device(s) and/or data storage device.

In some embodiments, the visual device 452 may include a computer system to carry out the image processing. The computer system may further be used to control the operation of the system or a separate system may be included.

The system 450 may include a computing system 460 capable of quantifying the force. In some embodiments, the computing system 460 may be a separate device. In other embodiments, the computing system 460 may be a part (e.g., stored on the memory) of other modules, for example, the visual device 452, and controlled by its respective CPUs.

The system 460 may be a computing system, such as a workstation, computer, or the like. The system 460 may include one or more processors (CPU) 462. The processor 462 may be one or more of any central processing units, including but not limited to a processor, or a microprocessor. The processor 462 may be coupled directly or indirectly to one or more computer-readable storage medium (e.g., physical memory) 464. The memory 464 may include one or more memory elements, such random access memory (RAM), read only memory (ROM), disk drive, tape drive, etc., or a combinations thereof. The memory 464 may also include a frame buffer for storing image data arrays. The memory 464 may be encoded or embedded with computer-readable instructions, which, when executed by one or more processors 462 cause the system 460 to carry out various functions.

In some embodiments, the system 460 may include an input/output interface 466 configured for receiving information from one or more input devices 472 (e.g., a keyboard, a mouse, joystick, touch activated screen, etc.) and/or conveying information to one or more output devices 474 (e.g., a printing device, a CD writer, a DVD writer, portable flash memory, display 476, etc.). In addition, various other peripheral devices may be connected to the computer platform such as other I/O (input/output) devices.

In some embodiments, the disclosed methods (e.g., FIG. 4A) may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### EXAMPLES AND METHODS

### Synthesis and Characterization of Streptavidin-Quencher Conjugates

A streptavidin labeling ratio of 1 was desired to accurately use the FRET relation and determine the zero-force conformation of the sensor. Recombinant streptavidin (Rockland Immunochemicals) was labeled with quencher by mixing 300 μg of the protein in 150 μl of 1×PBS (10 mM phosphate buffer, 137 mM NaCl, pH 7.4) with 15 μl of 1 M sodium bicarbonate and a 20-fold molar excess of QSY 21 N-hydroxysuccinimide (NETS) ester (Invitrogen). The reaction was allowed to proceed for 60 min at room temperature (23° C.) on a rotating platform. Purification was performed by size-exclusion chromatography using Bio-Gel P4 resin (Bio-Rad) swollen with 1×PBS. The final product was characterized using matrix-assisted laser desorption-time-of-flight (MALDI-TOF) and absorbance spectrometry. The labeling ratio was determined to be 0.8 by UV-visible light absorbance measurements of the gel-purified product.

Recombinant streptavidin was labeled with quencher by mixing 1 mg ml⁻¹ of the protein in 1×PBS with an excess of QSY 21 NHS ester. The reaction was allowed to proceed for 60 min at room temperature, and the tube was inverted every 15 min to ensure proper mixing. The product was purified with a Slide-a-Lyzer Mini dialysis column (Thermo Fisher) with a cutoff of 3,500 g mol⁻¹ following manufacturer recommendations and performing a 30 min dialysis in a 2-1 bath of 1×PBS twice. The final product was characterized using MALDI-TOF and absorbance spectrometry. Empirically, a fivefold molar excess of QSY 21 achieved a labeling ratio of ˜0.9-1.1. In contrast, a 20-fold molar excess of QSY 21 yielded streptavidin with a labeling ratio of ˜2 when using this method, based on UV-visible light absorbance measurements.

### Synthesis and Characterization of EGF-PEG Conjugates

EGF was simultaneously labeled with a flexible biotinylated PEG linker (PEG₁₂ (Thermo Scientific), PEG₂₄ (Quanta Biodesign) or PEG₇₅ (Nanocs)) and fluorescent dye (Alexa Fluor 647 (Invitrogen)) in a single pot reaction using standard NHS bioconjugation chemistry. A monolabeled product for both PEG and dye was desired for quantitative experiments. The optimal reaction concentrations were empirically determined to be 120 μM EGF, 0.1 M sodium bicarbonate and a fivefold molar excess of both the biotin-PEG NHS ester and the Alexa Fluor 647 NHS ester. The reaction was incubated on a rotating platform at room temperature for 30 min and purified using the Bio-Gel P6 resin (Bio-Rad). MALDI-TOF mass spectrometry and UV-visible light absorbance measurements were used to determine the overall EGF:PEG:dye ratio. Mass spectrometry indicated that the predominant product under these reaction conditions had an EGF:PEG:dye ratio of 1:1:1. Note that other EGF:PEG:dye stoichiometries existed in the sample, the most abundant of which was dual labeled with dye but not conjugated to the biotin-PEG anchor (1:0:2) and therefore would not adhere to the streptavidin-functionalized surfaces.

In some cases, EGF was labeled with biotinylated PEG₇₅ and Alexa Fluor 647 in a step-wise fashion. First, 10 μl of 1 M sodium bicarbonate was added to 100 μl of EGF (1 mg ml⁻¹), then 20-fold molar excess of Alexa Fluor 647 NHS ester was added and the reaction was allowed to proceed for 10 min at room temperature. Subsequently, a 15-fold molar excess of biotin-PEGS NHS ester was added to the reaction mixture and allowed to incubate for an additional 30 min. The reaction was purified using Bio-Gel P6 resin (Bio-Rad). The final labeling ratio of dye:protein, as measured by UV-visible light absorbance, was 0.8. The EGF that was used for the alkyl linker controls was labeled in a single-pot reaction with NHS-sulfo-LC-biotin (LC, long chain) (Pierce) and Alexa Fluor 647 NHS ester (Invitrogen). Sodium bicarbonate (20 μl of 1 M) was added to 200 μl of 1 mg ml⁻¹ EGF, after which a 20-fold molar excess of both biotinylated linker and dye was added. After reagent addition, the reaction was incubated for 1 h at room temperature and inverted every 15 min to ensure mixing. The reaction mixture was subsequently purified with Bio-Gel P4 resin (Bio-Rad), yielding EGF with an Alexa Fluor 647 labeling ratio of 1.9.

### Integrin-Specific Force Sensor

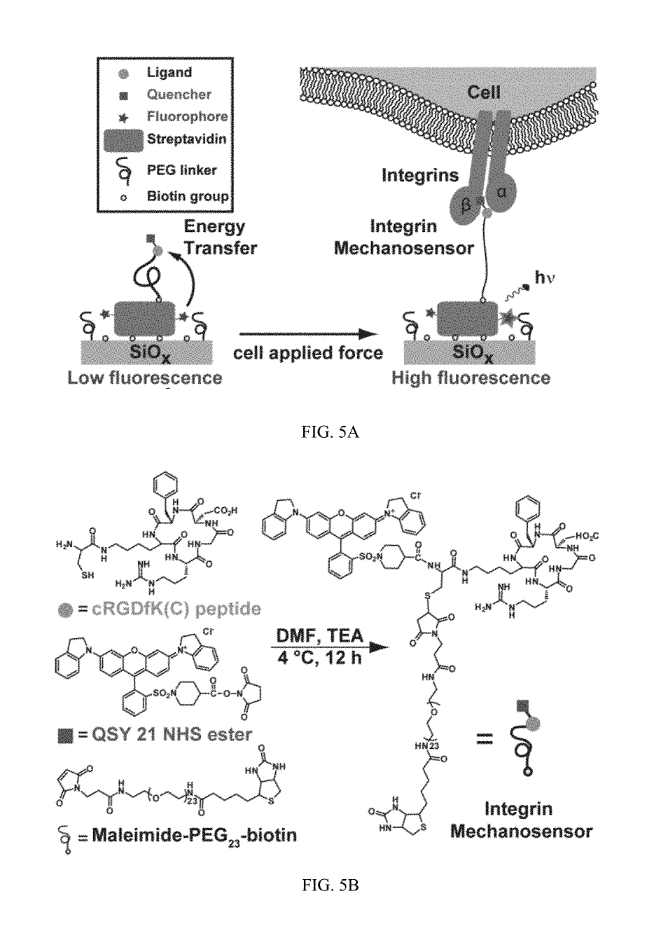

To specifically measure tension across the integrin receptors, a force sensor was created with a cyclic Arg-Gly-Asp (RGD) peptide analogue (FIG. 5B), which is a common motif found in ECM proteins, used as the ligand. The cyclic Arg-Gly-Asp-dPhe-Lys-Cys (cRGDfK(C)) peptide shows high affinity and selectivity towards αᵥβ₃ integrin receptor (αᵥβ₃>>α₅β₁ αᵥβ₅).

This force sensor is comprised of a 23 unit flexible polyethylene glycol (PEG) linker that reversibly extends in response to mechanical tension. One terminus of the PEG polymer is conjugated to the cRGDfK(C) peptide and a QSY 21 quencher, while the other terminus is conjugated to a biotin moiety (FIG. 5B). This peptide-PEG conjugate is then captured onto streptavidin-Alexa 647 that is immobilized onto a glass slide. Forces that are applied to the peptide will lead to its physical displacement away from a fluorophore-conjugated streptavidin, which results in de-quenching and an observed increase of fluorescence signal intensity. The quenching efficiency of the fluorescence signal is dependent on the inverse sixth power of the distance between the quencher and fluorophore. Accordingly, the magnitude of fluorophore de-quenching is used to measure the average polymer displacement (extension from equilibrium), which can, in turn, be used to quantify the minimum force applied between the integrin receptor and its cognate ligand.

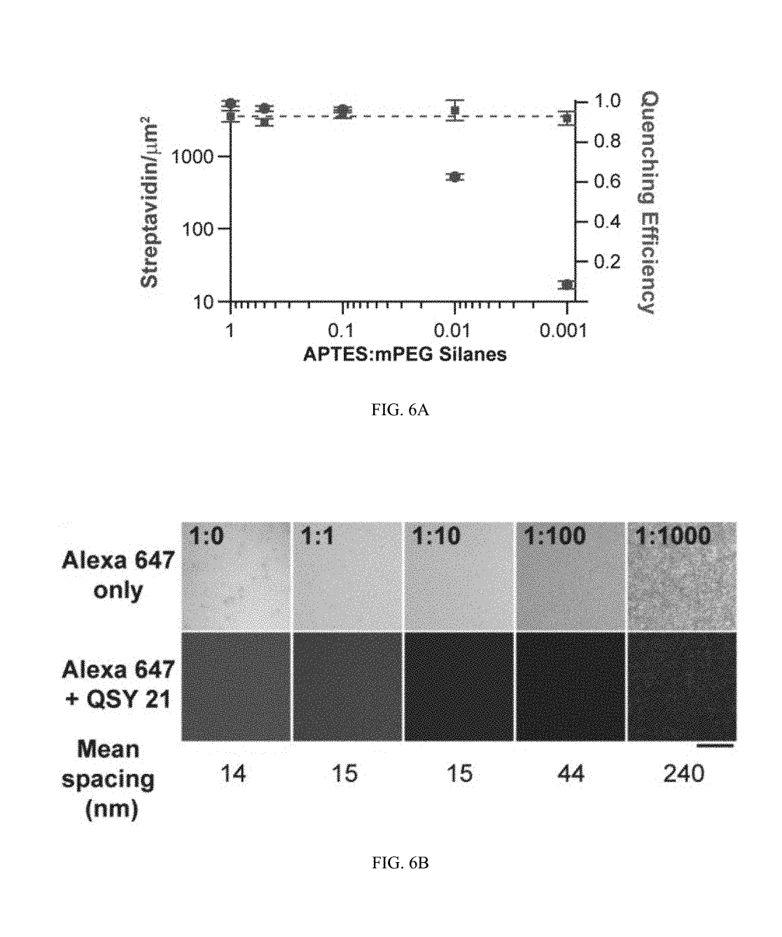

The sensor design has been modified such that the fluorophore is conjugated to the streptavidin, rather than the cRGD peptide, because integrins are known to assemble into clusters that would be expected to lead to self-quenching of RGD-fluorophores conjugates in close proximity. Given that the molecular density of the RGD peptide ligand dictates the formation and stability of cell adhesions, its surface density was measured and tuned. The RGD peptide surface density was controlled by incubating piranha-etched glass coverslips with a binary mixture of the two organosilanes: NH₂(CH₂)₃Si(OCH₂CH₃)₃ (APTES), and CH₃(CH₂CH═O)₉₋₁₂(CH₂)₃Si(OCH₃)₃ (mPEG). The terminal amine group of APTES was reacted with an NETS-ester biotin in order to capture streptavidin-Alexa647. In contrast, mPEG passivates the surface to minimize non-specific protein adsorption. To quantify the molecular surface density of the Peptide-PEG conjugate, the Alexa-647 fluorescence intensity was calibrated using a standard set of supported lipid membranes. This was achieved by synthesizing an Alexa-647 phospholipid conjugate, and doping it at low concentrations (<0.5 mol %) within a fluid 1,2-dioleoyl-sn-glycero-3-phosphocholine (DOPC) phospholipid bilayer. Based on this quantitative fluorescence calibration, APTES:mPEG ratios of 1:0, 1:1, 1:10, 1:100, and 1:1000 resulted in streptavidin surface densities of 5300, 4600, 4400, 520, and 17 molecules/μm², respectively (FIG. 6). Each streptavidin was conjugated to approximately one cRGD-peptide mechanophore when estimated to the nearest integer value (SI).

To determine the molecular conformation of the cRGD mechanophore, the average fluorophore-quencher distance was calculated using the FRET relation. In these experiments, the average fluorescence intensity of a surface that lacked the quencher was measured and compared to that of the mechanophore modified surfaces. The average quenching efficiency remained in a narrow range from 0.90 to 0.96 (average efficiency=0.93), thus indicating that the average fluorophore-quencher distance was 4.5 nm. This value is in agreement with the predicted Flory-Huggins model for a 23 unit PEG (Flory radius=2.3 nm, for a low-density grafted polymer in a good solvent), in addition to the radius of streptavidin (radius=2 nm, as estimated from the crystal structure [PDB:3ry1]). Moreover, the RGD surface density measurements also indicate that the average distance between any two streptavidin molecules is larger than the Flory radius, which confirms that the polymer should adopt the “mushroom” conformation on these surfaces. Therefore, the dynamic range of the RGD-integrin mechanophore is maintained across this range of molecular surface densities.

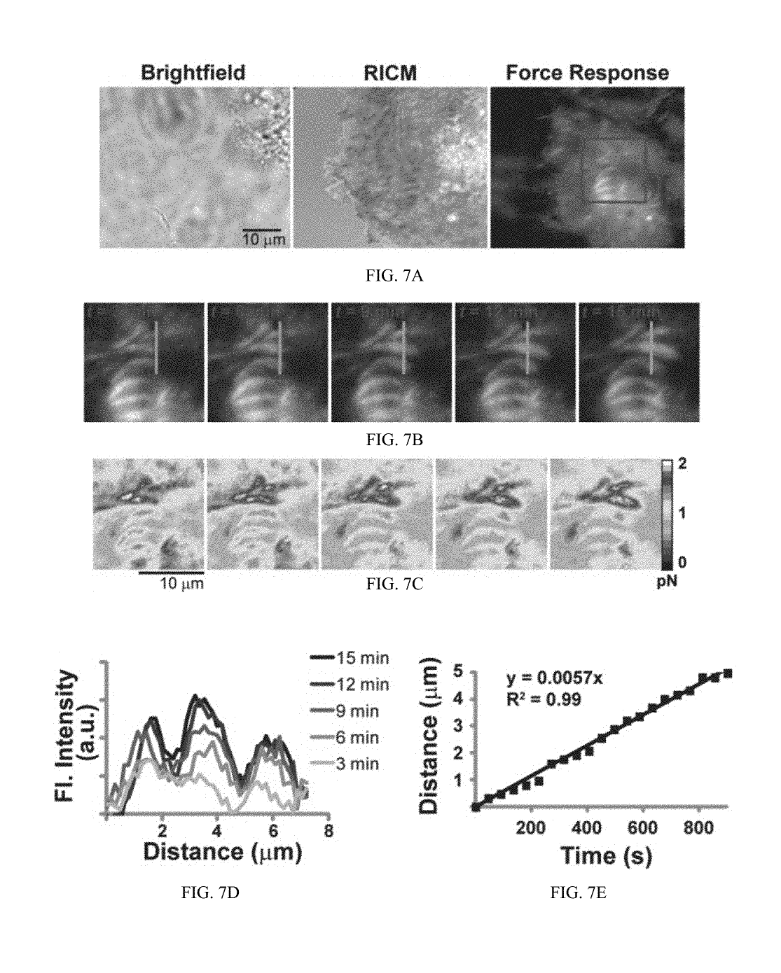

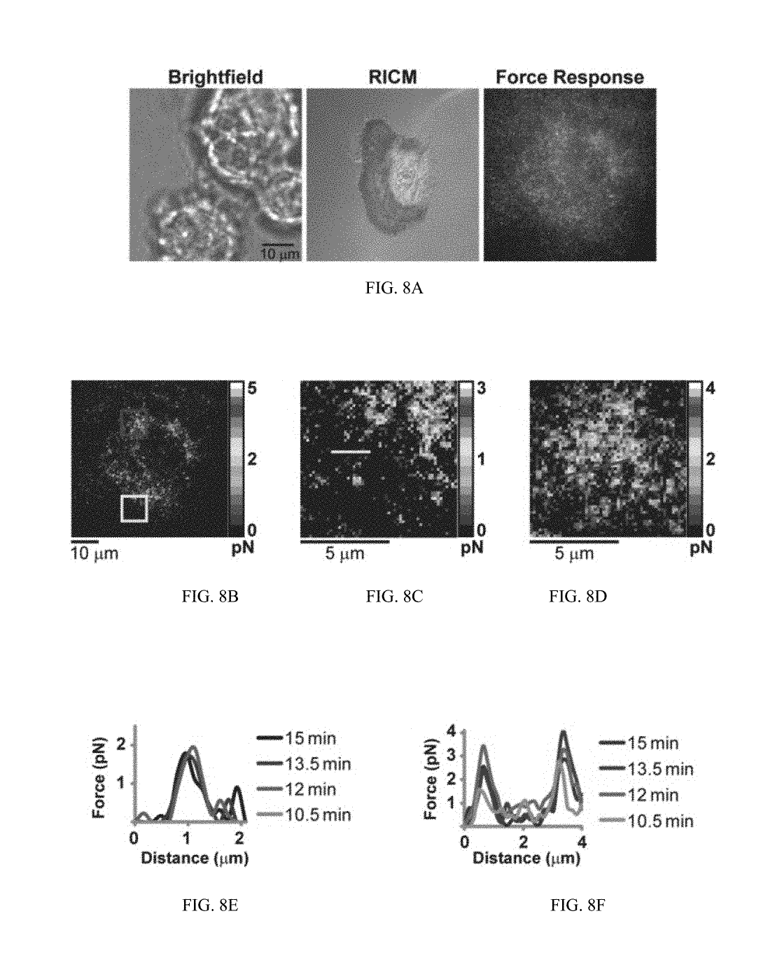

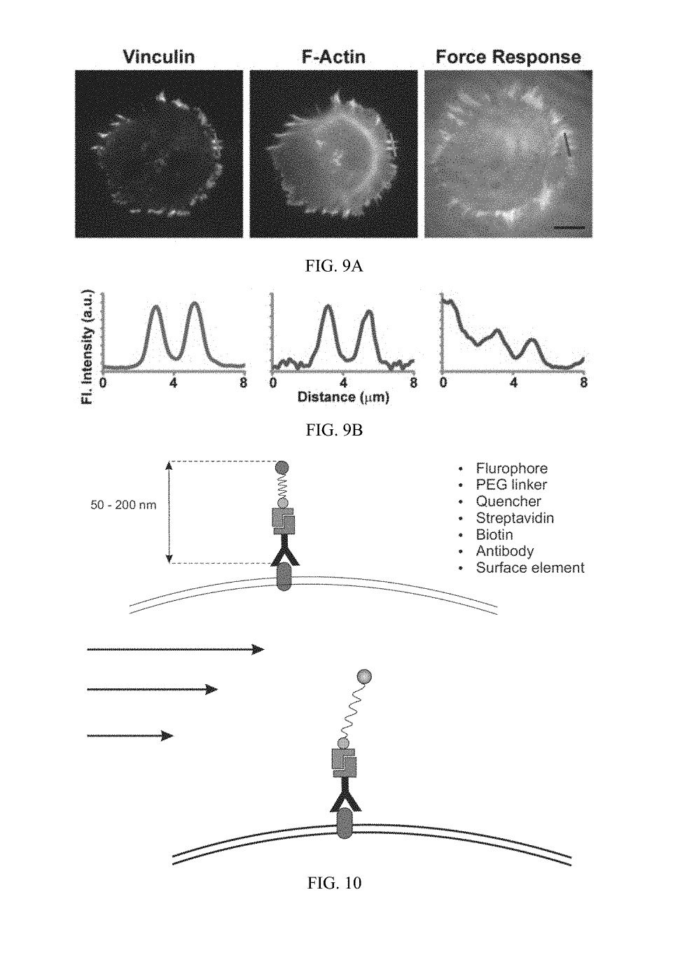

Multivalent or clustered RGD peptides are important to support the formation of focal adhesions. Therefore, in initial experiments, immortalized breast cancer cells (HCC1143) were directly plated onto the higher density RGD surfaces (4600±380 streptavidin molecules/μm²). After about 60 min of incubation at 37 C.° and 5% CO₂, the cells were imaged using brightfield, reflection interference contrast microscopy (RICM), and total-internal reflection fluorescence microscopy (TIRFM). The RICM channel revealed tight cell-substrate contact distances, whereas the TIRFM image shows subcellular regions that experience integrin-mediated mechanical tension (FIG. 7A). Time-lapse images that track the fluorescence intensity over a 15 min time duration revealed the dynamic growth of areas associated with integrin force transmission (FIG. 7B-D).

Quantitative force maps were generated from each fluorescence image using the extended worm-like chain model (FIG. 7C). Note that because this is an ensemble average measurement, the reported values in the quantitative force maps describe the lower bound estimate of force experienced by each integrin molecule. The force images reveal high aspect ratio, fiber-like, force regions that generally run parallel to each other. Interestingly, time-lapse force imaging shows the growth of the force regions with an average velocity of 0.006 μm/s (FIG. 7E).

Cells plated on surfaces with lower biotin densities exhibit a higher range of forces (up to 4 pN). This apparent increase in the applied force is likely the result of a larger fraction of cRGD mechanophore molecules that experience mechanical tension. Thus, force estimates on less dense surfaces will more accurately reflect the average tension applied per molecule. The magnitude of tension along each integrin molecule (˜1-4 pN) is in agreement with literature estimates based on elasticity theory data. Analysis of the integrin tension on these surfaces indicates that forces are more dynamic and short-lived than on surfaces with larger streptavidin density (>500 molecules/μm²). Additionally, cells are unable to form stable contacts with the substrate, as evidenced by the large fluctuation of cell-surface contact (seen in RICM). This observation is in agreement with previous work which tracked protrusion and retraction of the lamellipodium on surfaces with various RGD ligand densities.

As a control experiment, cRGDfK(C) surfaces were designed that lacked the quencher. With these conjugates, an extension of the PEG linker is not expected to lead to de-quenching. When HCC1143 cells were engaged to these control surfaces, no detectable increase in fluorescence intensity was observed. Finally, in order to verify that integrin tension correlates with focal adhesion formation, cells that were incubated on sensor surfaces, were subsequently fixed and immunostained for vinculin and filamentous-actin. A line plot analysis of the periphery of the cell shows that the force response co-localizes with focal adhesion related proteins. There are additional areas of tension, however, that do not associate with vinculin, and these may be related to “immature” focal adhesions.

### Cell Culture.

HCC1143 cells were cultured in RPMI 1640 medium (Mediatech) supplemented with 10% FBS (Mediatech), HEPES (9.9 mM, Sigma), sodium pyruvate (1 mM, Sigma), L-glutamine (2.1 mM, Mediatech), penicillin G (100 IU ml⁻¹, Mediatech) and streptomycin (100 μg ml⁻¹, Mediatech) and were incubated at 37° C. with 5% CO₂. Cells were passaged at 90-100% confluency and plated at a density of 50% using standard cell culture procedures. All experiments were conducted with HCC1143 cells that had been serum-starved for ˜18 h.

### Functionalization of Glass Substrate Biosensors

Glass coverslips (number 2, 25-mm diameter; VWR) were sonicated in Nanopure water (18.2 mΩ) for 10 min and then etched in piranha (a 3:1 mixture of sulfuric acid (Avantor Performance Materials) and hydrogen peroxide (Sigma)) for 10 min (please take caution: piranha is extremely corrosive and may explode if exposed to organics). The glass coverslips were then washed six times in a beaker of Nanopure water (18.2 mΩ) and placed into three successive wash beakers containing EtOH (Decon Labs) and left in a final fourth beaker containing 1% (3-aminopropyl)triethoxysilane (APTES, Sigma) in EtOH for 1 h. The substrates were then immersed in the EtOH three times and subsequently rinsed with EtOH and dried under nitrogen. Substrates were then baked in an oven (˜100° C.) for 10 min. After cooling, the samples were incubated with NHS-biotin (Thermo Fisher) at 2 mg ml⁻¹ in DMSO (Sigma) overnight. Subsequently, the substrates were washed with EtOH and dried under nitrogen. The substrates were then washed with 1×PBS (3×5 ml aliquots) and incubated with BSA (EMD Chemicals, 100 μg μl⁻¹, 30 min) and washed again with 1×PBS (3×5 ml aliquots). Quench labeled streptavidin was then added (1 μg ml⁻¹, 45 min, room temperature) followed by washing with 1×PBS (3×5 ml aliquots) and incubating with the desired EGF construct (biotinylated linker and fluorophore labeled, 1 μg ml⁻¹, 45 min, room temperature). Substrates were then rinsed with a final wash of 1×PBS (3×5 ml aliquots) and used within the same day. To verify that surfaces were stable within the experimental time frame, a substrate, functionalized as described above, was imaged over two consecutive days. The fluorescence intensity of the surface did not change greatly within this time frame.

### Functionalization of Supported Lipid Bilayers

Lipids consisted of 99.9% 1,2-dioleoyl-sn-glycero-3-phosphocholine (DOPC, Avanti Polar Lipids) and 0.1% 1,2-dioleoyl-sn-glycero-3-phosphoethanolamine-N-(cap biotinyl) (sodium salt) (DPPE-biotin, Avanti Polar Lipids). After being mixed in the correct proportions in chloroform, lipids were dried with a rotary evaporator and placed under a stream of N₂ to ensure complete evaporation of the solvent. These lipid samples were then resuspended in Nanopure water and subjected to three freeze-thaw cycles by alternating immersions in an acetone and dry ice bath and a warm water bath (40° C.). To obtain small unilamellar vesicle, lipids were extruded through a high-pressure extruder with a 100-nm nanopore membrane.

Supported lipid bilayers were assembled by adding small unilamellar vesicles to base-etched 96-well plates with glass-bottomed wells. At the biotinylated lipid doping concentration used (0.1%), the calculated streptavidin density was 690 molecules μm⁻², and therefore the streptavidin bound to the surface was at sufficiently low density to avoid fluorophore self-quenching. This was confirmed by measuring fluorescence intensity as a function of biotin doping concentration. After blocking with BSA (0.1 mg ml⁻¹) for 30 min, bilayer surfaces were incubated with either unlabeled streptavidin (1 μg 400 μl⁻¹) or streptavidin QSY 21 (1 μg 400 μl⁻¹) for 1 h. Wells were rinsed 3× with 5 ml of 1×PBS, then incubated with EGF-PEGₓ-Alexa Fluor 647 (100 nM) (x=12, 24 or 75) for 1 h and rinsed 3× with 5 ml of 1×PBS before imaging.

### Characterization of the Zero-Force Sensor Conformation

FRET efficiency was measured using equation (1)

$$E = ( 1 - \frac{{I}_{DA}}{{I}_{D}} ) \frac{1}{{f}_{A}} \qquad ( 1 )$$

where I_{DA} refers to the intensity of the EGF-PEGₓ-Alexa Fluor 647 surface containing quench labeled streptavidin, I_{D} is the intensity of the EGF-PEGₓ-Alexa Fluor 647 surface with unlabeled streptavidin and f_{A} is the labeling ratio of the acceptor. These values were obtained by averaging the fluorescence intensity measured in five different areas for each substrate. The reported values are the average of three independent experiments. The calculated efficiency for each surface was then used to determine the average distance between fluorophore and quencher by,

$$E = \frac{1}{1 + {( \frac{r}{{R}_{0}} )}^{6}} \qquad ( 2 )$$

where R₀ is the Förster distance of the dye pair (6.9 nm according to the manufacturer) and r is the average distance between the fluorophores. The predicted value for r was determined by adding the PEG Flory radius to the radii of the proteins that comprise the force sensor. The protein radius for EGF was estimated at 1 nm based on its crystal structure (Protein Data Bank (PDB): 2KV4), and for streptavidin the radius was estimated at 2 nm based on the crystal structure (PDB: 1SWB). The predicted r value was then compared to the FRET measured r value.

### Fluorescence Microscopy

Live cells were imaged in serum-free RPMI 1640 (Mediatech) medium formulated as described in the cell culture section at 37° C., and fixed cells were imaged in 1% BSA in 1×PBS at room temperature. During imaging, physiological temperature was maintained with a warming apparatus consisting of a sample warmer and an objective warmer (Warner Instruments 641674D and 640375). The microscope used was an Eclipse Ti driven by the Elements software package (Nikon). The microscope features an Evolve electron multiplying charge-coupled device (CCD; Photometrics), an Intensilight epifluorescence source (Nikon) a CFI Apo 100×(numerical aperture (NA) 1.49) objective (Nikon) and a TIRF launcher with two laser lines: 488 nm (10 mW) and 640 nm (20 mW). This microscope also includes the Nikon Perfect Focus System, an interferometry-based focus lock that allowed the capture of multipoint and time-lapse images without loss of focus. The microscope was equipped with the following Chroma filter cubes: TIRF 488, TIRF 640, Cy5 and reflection interference contrast microscopy (RICM).

### Image Analysis

Images from sensor experiments were processed (using a custom macro in ImageJ (US National Institutes of Health)) from a single multipoint image file into individual tiff stacks containing each imaging channel. Separate macros were then used to isolate and background subtract the Alexa Fluor 647 EGF force channel. For all images, the LUT was linear and represented the full range of data as indicated by the calibration bar accompanying each image set. Analysis of images was performed with ImageJ and Nikon Elements software packages. ND2 image processing was done with several custom imageJ macros in combination with the LOCI bio-formats ImageJ plugin as well as the Nikon Elements software package. Sensor spot duration analysis was performed manually with the assistance of the SpotTracker 2D and Multi Measure ImageJ plugins.

### Quantitative Force Maps

To determine the absolute magnitude of forces detected by the sensor, a series of image operations were performed. First, the quenching efficiency image map was derived from the background subtracted TIRF 640 sensor signal image by using equation (3)

$$C = 1 - \frac{A}{B} \qquad ( 3 )$$

where A is the background-subtracted TIRF 640 sensor signal image, B is the average background-subtracted TIRF 640 image of a donor-only force probe obtained from a sample lacking the quencher and C is the resulting image which is a map of the quenching efficiency. Next, an image mapping the distance between the fluorophore and quencher was obtained by rearranging the FRET relation and applying equation (4)

$$D = {{R}_{0} ( \frac{1}{C} - 1 )}^{1 / 6} \qquad ( 4 )$$

where R₀ is the Förster radius of the quencher-fluorophore pair, and D is the resulting distance map. This fluorophore-quencher distance image was then used to correct for the TIRF excitation intensity because the evanescent field intensity drops off exponentially in the z axis dimension. The penetration depth of the TIRF evanescent field was determined by equation (5)

$$d = \frac{λ}{4 π \sqrt{{n}_{2}^{2} {sin}^{2} θ - {n}_{1}^{2}}} \qquad ( 5 )$$

where d is the penetration depth of the evanescent field, n₂ is the index of refraction of glass (1.51), n₁ is the index of refraction of water (1.33), λ is the wavelength (640 nm) and θ is the incident angle of the laser (˜65°). The penetration depth can then be used along with the distance map to determine the corrected TIRF excitation intensity at each pixel. This is accomplished by applying equation (6)

S=Be^{-D/d} (6)

where S is the scalar correction image, B is the donor only averaged image, D is the distance map image and d is the penetration depth of the evanescent field. The product of multiplying S by B gives the illumination intensity corrected distance map, E. To determine the average PEG resting conformation, the dimensions of EGF and streptavidin were subtracted from the corrected distance map, E. To calculate the extension of PEG from this resting state, the PEG resting state conformation was subtracted from the entire image. Finally, a quantitative force map was inferred by applying the extended WLC model to the distance map. The extended WLC approximation is made by applying equation (7) to image E

$$F = \frac{{k}_{B} T}{{L}_{p}} ( \frac{E}{L} + \frac{1}{4 {( 1 - \frac{E}{L} )}^{2}} - \frac{1}{4} + {\sum }_{i = 2}^{i = 7} {{α}_{i} ( \frac{E}{L} )}^{i} ) \qquad ( 7 )$$

where F is the resulting quantitative force map image, k_{B} is the Boltzman constant, T is the temperature, Lₚ is the persistence length of PEG (0.38 nm), E is the corrected distance map and L is the end-to-end length of PEG₂₄ (8.4 nm).

### Determination of EGFR Phosphorylation and Activation

HCC1143 cells were seeded onto the biosensor surfaces displaying EGF and incubated on the substrates for 30 min at 37° C. Following initial imaging, the cells were fixed with 4% paraformaldehyde (Sigma) in 1×PBS and permeabilized with 0.1% Triton X (Sigma) in 1×PBS. Cells were then blocked overnight in 1% BSA at 4° C. The next day, cells were incubated with a primary antibody to EGFR-pTyr1068 (Cell Signaling Technologies 3777s) at 1:200 dilution for 1 h at room temperature. The primary antibody was then washed out with 1×PBS and the cells were incubated with Alexa Fluor 488-labeled rabbit IgG secondary antibody (Invitrogen) at 1:1,000 dilution for 45 min. The secondary antibody was then rinsed out with 1×PBS, and the sample was imaged in TIRF mode at 488 nm as well as in the Alexa Fluor 647, brightfield and RICM channels using an epifluorescence source.

### Actin Inhibition

HCC1143 cells were serum-starved for ˜18 h and split into two aliquots, one of which was treated with 4 μm latrunculin B (Sigma) for 30 min in DMSO (EMD Chemicals), and the other was treated with an equivalent amount of DMSO. Each aliquot was then plated onto an EGF-functionalized biosensor surface and incubated for 30 min at 37° C. Cells were then imaged in the Alexa Fluor 647, brightfield and RICM channels.

### CLC-eGFP Transfection

HCC1143 cells were seeded on a 24-well plate in antibiotic-free media at a density of 300,000 cells per well overnight. The cells were then transfected with the CLC-eGFP construct using Lipofectamine 2000 (Invitrogen) and following standard transfection protocols. These cells were then serum-starved overnight and used for experiments as indicated within 24 h of the transfection.

What we claim:

1. A method of detecting a light signal from a cell receptor binding a ligand comprising the steps of:
a) exposing a device to a cell containing a receptor to the ligand under conditions such that the device is connected to the cell membrane comprising the receptor of the ligand;
wherein the device comprises:
i) a ligand;
ii) an elastic linker molecule having a first end and a second end, wherein the linker is linked to the ligand at the first end;
iii) a surface connected to the elastic linker at the second end;
iv) a first fluorescent molecule conjugated to the ligand wherein the fluorescent molecule is configured to move its position relative to the surface when the ligand moves; and
v) a quencher fixed to the surface,
wherein the elastic linker comprises ethylene glycol, polypeptide, or polynucleotide; and
wherein the receptor is binding and pulling the ligand away from the surface to extend the elastic linker from the relaxed conformation removing the first fluorescent molecule from proximity to the quencher producing a light signal; and
b) detecting the light signal.

2. The method of claim 1, wherein the extended length of the linker from the relaxed conformation is greater than 10 nm.

3. The method of claim 1, wherein the linker is further connected to the surface through a polypeptide.

4. The method of claim 1, wherein the polypeptide is conjugated to the quencher.

5. The method of claim 1, wherein the polypeptide is streptavidin or an antibody.

6. The method of claim 1, wherein the ligand is a biological molecule, protein, protein fragment, nucleic acid, glycoprotein, polysaccharide, hormone, steroid, therapeutic agent, or other molecule with affinity for a protein or receptor.

7. The method of claim 1, wherein the surface is a particle, bead, wafer, array, well, microscope slide, or bottom of a zero-mode waveguide.

8. The method of claim 1, wherein the first fluorescent molecule is a fluorescent dye, quantum dot, or fluorescent protein.
